(19) United States
(12) Patent Application Publication
(10) Pub. No.: US 2023/0411007 A1
(43) Pub. Date: Dec. 21, 2023

(54) INTELLIGENT AUXILIARY GOUT DIAGNOSIS AND TREATMENT SYSTEM FOR COMBINATION OF TRADITIONAL CHINESE MEDICINE AND WESTERN MEDICINE

(71) Applicants: Zhejiang Chinese Medical University, Hangzhou (CN); XinTong Research Institute of Artificial Intelligence, Yuhang, Hangzhou, Hangzhou (CN)

(72) Inventors: Chengping Wen, Hangzhou (CN); Lin Huang, Hangzhou (CN); Mingzhi Zheng, Hangzhou (CN); Zhijun Xie, Hangzhou (CN)

(21) Appl. No.: 18/310,709

(22) Filed: May 2, 2023

(30) Foreign Application Priority Data
Jun. 21, 2022 (CN) 2022107061640

(51) Int. Cl. G16H 50/20; G16H 20/90; G06N 5/04

(52) U.S. Cl. CPC G16H 50/20; G16H 10/60; G06N 5/04; G16H 20/90

(57) ABSTRACT

An intelligent auxiliary gout diagnosis and treatment system for combination of traditional Chinese medicine and western medicine includes a knowledge extraction module, a predictive reasoning module, an evaluation feedback module and a data storage module. The knowledge extraction module is configured to construct a gout knowledge graph. The predictive reasoning module is configured to learn a predictive model in combination with historical annotation data to perform reasoning diagnosis, predict a gout course stage of a patient and recommend a treatment plan. The evaluation feedback module is configured to evaluate a diagnosis and treatment effect for strengthening the system and improving an intelligent level of the system. The data storage module is configured to store data of the system.

### TECHNICAL FIELD

The disclosure relates to the technical field of intelligent auxiliary gout diagnosis and treatment, in particular to an intelligent auxiliary gout diagnosis and treatment system for combination of traditional Chinese medicine (TCM) and western medicine (WM).

### BACKGROUND

With the development of science, people are eager to introduce the big data technology and the artificial intelligence technology into the medical field to empower the medical industry. At present, it is of more practical significance to research and develop intelligent medical products for special diseases, and the medical level of the special diseases can be improved and the related research can be further promoted by collecting specialist knowledge and constructing intelligent systems.

Gout is a chronic treatable disease caused by the deposition of monosodium urate crystals (i.e., tophus) in joints and non joint structures, which occurs intermittently. A natural course of the gout can be divided into 3 stages: (i) asymptomatic hyperuricemia; (ii) recurrent attacks of acute gouty arthritis with asymptomatic during interval; (iii) chronic gouty arthritis, usually with obvious tophus at this stage. The most important risk factor in development of the gout is elevated serum urate concentration (hyperuricemia). Rheumatology Association in the United States, European and China have formulated many gout diagnosis and treatment guidelines to guide clinical treatment with uric acid-lowering drugs and anti-inflammatory and analgesic drugs, but its disadvantage is the high number of side effects and complications. Some studies have shown that the combination of TCM and WM has unique advantages of efficiency and toxicity reduction when treat the gout. However, due to the lack of veteran TCM specialists, the treatment of the combination of TCM and WM for the gout has not been widely used.

In view of the current serious diagnosis and treatment situation of the gout, it is urgent to construct a professional intelligent auxiliary diagnosis and treatment system. Constructing the intelligent auxiliary diagnosis and treatment system for the gout treatment faces many challenges: First, it lacks an intelligent diagnosis and treatment system architecture that can be directly learned and used; Second, there are many TCM classics related to the gout, but it is difficult to extract knowledge from the TCM classics because of the lack of simple and feasible diagnosis and treatment guidelines; third, simple TCM diagnosis and treatment methods have been difficult to meet the needs of the modern medical development, and it is difficult to organically combine TCM and WM in the intelligent system.

Constructing the intelligent auxiliary gout diagnosis and treatment system through introducing artificial intelligence technology can reduce the dependence on the TCM specialists and improve the level of gout diagnosis and treatment. In addition, it further provides the support for the research on the gout treatment with TCM, which has very important practical application value and theoretical significance.

### SUMMARY

The disclosure provides an intelligent auxiliary gout diagnosis and treatment system for combination of TCM and WM, which enables the user (i.e., doctor) to input patient's symptoms to quickly obtain a diagnosis result and recommended treatment plan, the intelligent auxiliary gout diagnosis and treatment system can continuously track and evaluate patient's effect after the treatment, and enhance the system intelligence level through reinforcement learning.

The disclosure describes the intelligent auxiliary gout diagnosis and treatment system for combination of TCM and WM includes the following modules: a knowledge extraction module, a predictive reasoning module, an evaluation feedback module and a data storage module. The knowledge extraction module is configured to extract correlative information from existing ancient books, Chinese and foreign language literatures and treatment guidelines to construct a knowledge graph of gout. The predictive reasoning module has an offline training stage and an online use stage, the predictive reasoning module is configured to perform model training to obtain a predictive model by using annotated medical data and the knowledge graph in the offline training stage, and the predictive reasoning module is configured to receive inputted symptoms of a patient and a western medical test result to perform reasoning diagnosis and predict a gout course stage of the patient, recommend a treatment plan, and output a treatment case with a highest similarity to the symptoms of the patient in a database in the online use stage. The evaluation feedback module is configured to collect specialist diagnostic advices and treatment effects on the patient, and adjust the predictive model in the predictive reasoning module through reinforcement learning according to the specialist knowledge and treatment evaluation results. The data storage module is configured to store system data, the system data includes correlative literature resources and the knowledge graph in the knowledge extraction module, training data required in the predictive reasoning module and predictive model files generated in the predictive reasoning module, patient input data, specialist diagnosis results and post-treatment evaluation data in the evaluation feedback module.

An intelligent auxiliary gout diagnosis and treatment system for combination of TCM and WM, includes: a knowledge extraction module, a predictive reasoning module, an evaluation feedback module and a data storage module.

The knowledge extraction module is configured to extract correlative information from existing ancient books, Chinese and foreign language literatures and treatment guidelines to construct a knowledge graph of gout.

The predictive reasoning module has an offline training stage and an online use stage, the predictive reasoning module is configured to perform model training to obtain a predictive model by using annotated medical data and the knowledge graph in the offline training stage, and the predictive reasoning module is configured to receive inputted symptoms of a patient and a western medical test result to perform reasoning diagnosis and predict a gout course stage of the patient, recommend a treatment plan, and output a treatment case with a highest similarity to the symptoms of the patient in a database in the online use stage.

The evaluation feedback module is configured to collect specialist diagnostic advices and treatment effects on the patient, and adjust the predictive model in the predictive reasoning module through reinforcement learning according to the specialist knowledge and treatment evaluation results.

The data storage module is configured to store system data, the system data includes correlative literature resources and the knowledge graph in the knowledge extraction module, training data required in the predictive reasoning module and predictive model files generated in the predictive reasoning module, patient input data, specialist diagnosis results and post-treatment evaluation data in the evaluation feedback module.

In an embodiment, the data storage module includes a memory. The knowledge extraction module is embodied by at least one processor and at least one memory coupled to the at least one processor, and the at least one memory stores programs executable by the at least one processor. Likewise, the predictive reasoning module is embodied by at least one processor and at least one memory coupled to the at least one processor, and the at least one memory stores programs executable by the at least one processor. Likewise, the evaluation feedback module is embodied by at least one processor and at least one memory coupled to the at least one processor, and the at least one memory stores programs executable by the at least one processor.

In an embodiment, the knowledge extraction module is configured to perform literature knowledge extraction, specialist knowledge extraction and guideline knowledge extraction.

In an embodiment, the predictive reasoning module is configured to perform offline learning training, similar case presentation, staged diagnostic output and TCM prescription recommendation

In an embodiment, the evaluation feedback module is configured to perform specialist follow-up diagnostics feedback and treatment effect evaluation feedback.

In an embodiment, the data storage module is configured to perform knowledge graph storage, training model storage and original data storage.

In an embodiment, the knowledge extraction module is configured to extract correlative information from existing ancient books, Chinese and foreign language literatures and treatment guidelines to construct a knowledge graph of gout, and the knowledge extraction module includes a model layer and a data layer.

The model layer is configured to define an ontology type by TCM specialists, the ontology type includes named entity classification and entity relationship classification (i.e., the model layer is configured to name the entity classification and entity relationship classification).

The data layer is configured to perform manual annotation on extracted electronic medical record data to obtain annotated samples, and perform automatic annotation on electronic medical records by using the annotated samples and a sequence annotation algorithm to identify entities and entity relationships in medical materials, and store the entities and the entity relationships into the database.

In an embodiment, the predictive model of the predictive reasoning module is configured to divide a gout course to obtain gout course stages.

In an embodiment, in the offline training stage of the predictive reasoning module, the predictive model is obtained by training a data set containing input symptom, corresponding syndrome elements of disease nature, and corresponding syndrome elements of disease locations.

In the online use stage, the predictive reasoning module is configured to receive patient data, and extract information for determining the gout course stage including a serum urate concentration, locations and numbers of joint swelling and pain and extract information for the symptoms of the patient by using keyword, synonym matching and semantic understanding technology, evaluate the gout course stage based on the extracted information, and match a basic diagnosis and treatment plan for the gout course stage; the predictive reasoning module is configured to use the predictive model to predict syndrome elements of disease nature and disease locations according to the symptoms of the patient, search from the knowledge graph according to the predicted syndrome elements of the disease nature and the disease locations to obtain TCM drugs; and form the recommendatory treatment plan by combining the basic treatment plan and the TCM drugs.

In the disclosure, the intelligent auxiliary diagnosis and treatment system includes the knowledge extraction module, the predictive reasoning module, the evaluation feedback module and the data storage module. The knowledge extraction module is configured to construct the knowledge graph of gout. The predictive reasoning module is configured to learn the predictive model in combination with historical annotation data to perform reasoning diagnosis, predict a gout course stage of a patient and recommend a treatment plan. The evaluation feedback module is configured to evaluate a diagnosis and treatment effect for strengthening the system and improving an intelligent level of the system. The data storage module is configured to store data of the system. The complexity of gout is considered and multilevel gout related knowledge are mined in the disclosure, and the disclosure combines TCM and WM to diagnose and predict, and constructs the evaluation feedback module, so as to greatly improve reliability and intelligence of the gout auxiliary diagnosis and treatment system.

Compared with the related art, the disclosure has the following advantages:

(1) knowledge mining is performed from multilevel for the complex problem of gout, the knowledge include the TCM ancient books, the Chinese and foreign related literature and the specialist knowledge; (2) when the diagnosis reasoning system is constructed, on the one hand, the gout course is divided into different stages based on existing medical data, so as to achieve staging refinement predictive; on the other hand, symptom representations of TCM is combined with WM test results to perform learning training of predictive model, the disclosure further improves the accuracy of model prediction; furthermore, in addition to outputting diagnostic results, a similarity matching model is constructed to output a similar treatment case from historical cases; (3) a feedback evaluation mechanism is constructed, and based on the specialist diagnosis results combination and the follow-up evaluation, intelligence level of system is enhanced by using new patient's treatment data.

### BRIEF DESCRIPTION OF DRAWINGS

FIG. 1 is a schematic diagram of an intelligent auxiliary gout diagnosis and treatment system for combination of TCM and WM according to an embodiment of the disclosure.

FIG. 2 is a schematic diagram of relationships among modules in the intelligent auxiliary gout diagnosis and treatment system for combination of TCM and WM according to an embodiment of the disclosure.

FIG. 3 is a schematic diagram showing a construction of a knowledge graph.

FIG. 4 is a schematic diagram showing an example of a knowledge graph.

FIG. 5 is schematic diagram showing a workflow of a predictive reasoning module.

### DETAILED DESCRIPTION OF EMBODIMENTS

The following is a detailed description of an intelligent auxiliary gout diagnosis and treatment system for combination of TCM and WM with the accompanying drawings and embodiments of the disclosure.

(1) An overall structure of the intelligent auxiliary gout diagnosis and treatment system for combination of TCM and WM

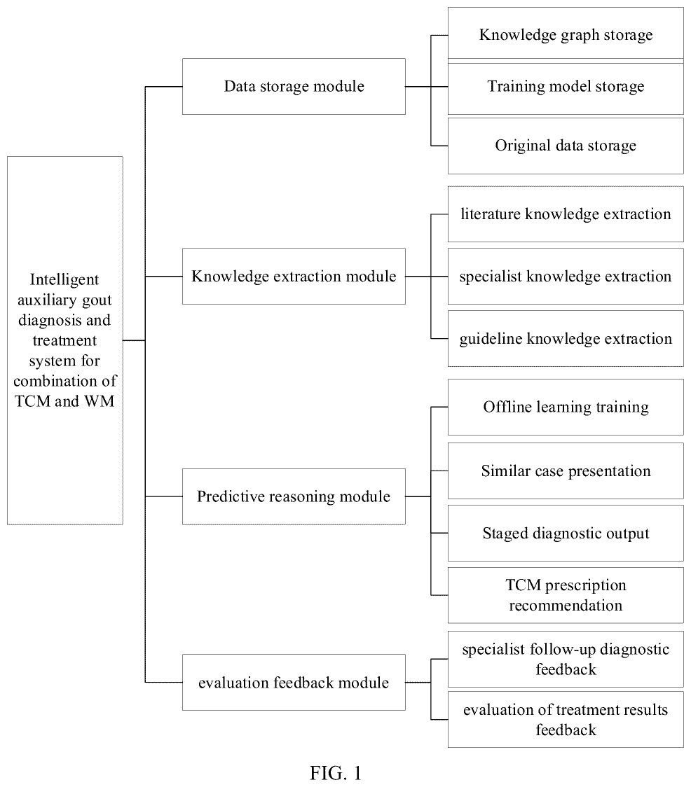

In the new era, a gout diagnosis and treatment decision support system based on artificial intelligence is still blank, so it is urgent to build such the system to instruct young doctors and grassroots doctors to diagnose and treat gout more accurately, thereby to improve clinical efficacy of gout and promote relevant research of gout. For this purpose, the disclosure provides an intelligent auxiliary gout diagnosis and treatment system for combination of TCM and WM, as shown in FIG. 1, the system includes a knowledge extraction module, a predictive reasoning module, an evaluation feedback module and a data storage module. The data storage module includes a knowledge graph storage, a training model storage and an original data storage. The knowledge extraction module includes a literature knowledge extraction, a specialist knowledge extraction and a guideline knowledge extraction. The predictive reasoning module incudes an offline learning training, a similar case presentation, staged diagnostic output and TCM prescription recommendation. The evaluation feedback module (i.e., feedback enhancement module) includes specialist follow-up diagnostic feedback and treatment effect evaluation feedback.

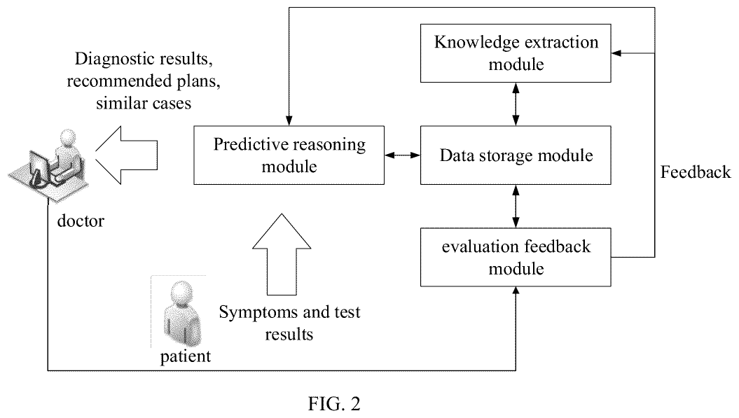

The module relationships and a main use process are shown in FIG. 2. The knowledge extraction module is configured to construct a knowledge graph of gout by using multi-layered information and store the knowledge graph of gout which as a priori knowledge for subsequent modules. The predictive reasoning module is a core part of the system, and the predictive reasoning module is configured to train a model using a large amount of annotated clinical data based on the above priori knowledge to diagnose conditions of a patient, recommend a treatment plan and display similar treatment cases in the database. During actual use, the predictive reasoning module is configured to receive patient-related information, and output the predictive result to the user (i.e., doctor). The evaluation feedback module is configured to perform feedback correction through specialist diagnosis and long-term follow-up evaluation, so as to update the predictive reasoning module and knowledge extraction module and realize the system enhancement. The data storage module is configured to provide data storage and data change to the system, the storage content of the data storage module includes following three parts: original data, structuration knowledge graph data after knowledge extraction and module data of learning training, the data storage module has data interaction with each module.

The following describes embodiments of the knowledge graph construction, staging diagnosis model and predictive reasoning module.

(1) Knowledge Graph Construction:

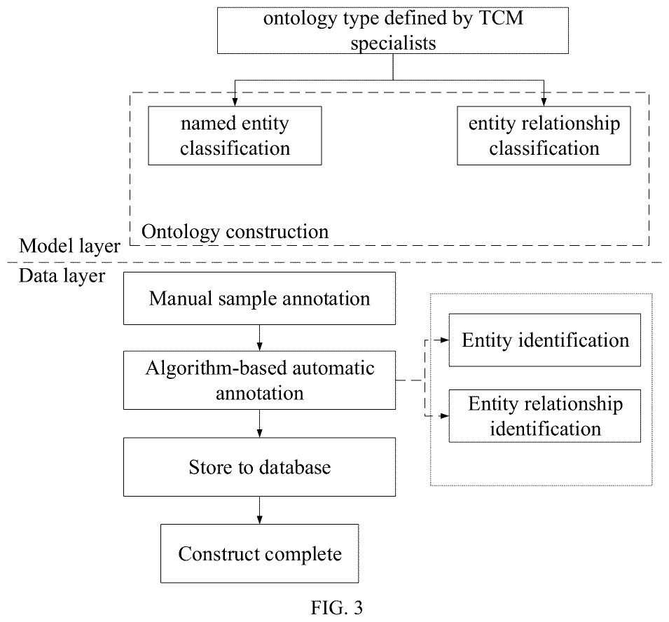

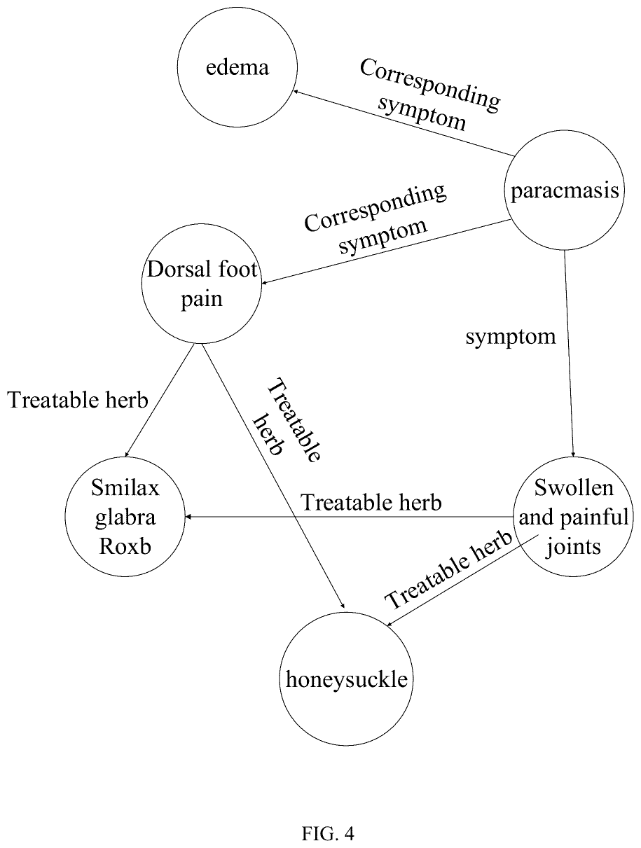

As shown in FIG. 3, the TCM specialist defines a type of an ontology at first, the ontology includes named entity classification (such as disease, symptom, treatment and prescription etc.), entity relationship classification (such as disease—include—symptom and prescription—treatment—symptom etc.), the named entity classification and the entity relationship classification form a model layer of the knowledge graph, then extracted small-scale electronic medical record data are manually annotated. The large-scale electronic medical record is automatically annotated through using the annotation small-scale samples and a sequence labeling algorithm such as conditional random fields (CRF) or hidden Markov model (HMM), so as to identify the entities and entity relationships in medical materials, and then store the entities and entity relationships into the database. In the knowledge fusion part, the problems of entity ambiguity and co-reference are solved through using context. The entities and their relationships form the data layer of the knowledge graph. An example of the knowledge graph presentation is shown in FIG. 4.

(2) Establish the Staging Diagnosis Model:

According to the laboratory examination and the patient's clinical manifestations, the course of gout is divided to four stages, as shown in table 1. The different treatment plans are used in different stages, after the multi-stage division, the refined diagnostic level of the system can be improved, so as to achieve better apply the medicine to the symptom.

Table 1 illustrates a stage division of gout course

| Stage | Division criteria |
|---|---|
| Asymptomatic | Serum urate level is higher than 6.8 mg/dl, |
| Hyperuricemia | and arthritis or uric acid kidney stones are |
| | not presented |
| Acute Gouty Arthritis | Arthritic episodes, involvement of the first |
| | metatarsophalangeal joint is common, and |
| | monosodium urate crystals are detected in |
| | synovial fluid leukocytes |
| Interval Gout | The stage between gout attacks |
| Chronic Gouty | Chronic non-intermittent polyarthritis with |
| Arthritis | urate deposits |

Based on the Entity and entity relationship data completed by manual and automatic annotation in the previous step, data mining algorithms such as Apriori, Frequent pattern growth (FP-growth) are used to mine cases belonging to different stages of gout course to obtain each stage basic diagnosis and treatment plans (include the TCM and WM).

(3) The Predictive Reasoning Module:

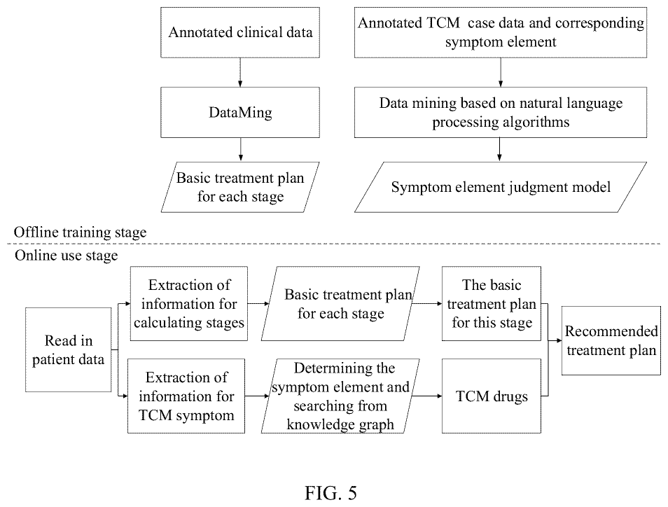

As shown in FIG. 5, in the online use stage, the predictive reasoning module is configured to receive patient data, and extract information for determining the gout course stage including a serum urate concentration, locations and numbers of joint swelling and pain and extract the symptoms of the patient by using keyword, synonym matching and semantic understanding technology, evaluate the gout course stage based on the extracted information, and match a basic diagnosis and treatment plan for the gout course stage. The predictive reasoning module is further configured to use the syndrome element predictive model to predict syndrome elements of disease nature and disease locations according to the symptoms of the patient., The syndrome element predictive model is a classification predictive model obtained by training a data set containing input symptoms, corresponding syndrome elements of disease nature, and corresponding syndrome elements of disease locations. The predictive reasoning module is further configured to search from the knowledge graph according to the predicted syndrome elements of the disease nature and the disease locations to obtain TCM drugs; and form the recommendatory treatment plan by combining the basic treatment plan and the TCM drugs, as a reference for doctors.

(4) The Evaluation Feedback Module:

The evaluation feedback module is configured to adjust and enhance the system by using new patient's information. The feedback evaluation mechanism comes from two parts, the first part is that the specialists improve or correct the predictive results and the recommended plans after the predictive reasoning module gives the prediction; the second part is to establish a long-term follow-up concern for patients, regular evaluate the treatment effect of the patient, and dynamically adjust the weight of the recommended treatment plan according to the treatment effect.

What is claimed is:

1. A gout diagnosis and treatment system for combination of traditional Chinese medicine (TCM) and western medicine (WM), comprising:
a knowledge extraction module, configured to extract correlative information from existing ancient books, Chinese and foreign language literatures and treatment guidelines to construct a knowledge graph of gout;
a predictive reasoning module, configured to perform model training to obtain a predictive model by using annotated medical data and the knowledge graph in an offline training stage, and the predictive reasoning module is configured to receive inputted symptoms of a patient and a western medical test result to perform reasoning diagnosis and predict a gout course stage of the patient, recommend a treatment plan, and output a treatment case with a highest similarity to the symptoms of the patient in a database in an online use stage;
an evaluation feedback module, configured to collect specialist diagnostic advices and treatment effects on the patient, and adjust the predictive model in the predictive reasoning module through reinforcement learning according to specialist knowledge and treatment evaluation results; and
a data storage module, configured to store system data, wherein the system data comprises correlative literature resources and the knowledge graph in the knowledge extraction module, training data required in the predictive reasoning module and predictive model files generated in the predictive reasoning module, patient input data, specialist diagnosis results and post-treatment evaluation data in the evaluation feedback module.

2. The gout diagnosis and treatment system for combination of TCM and WM according to claim 1, wherein the knowledge extraction module is configured to perform literature knowledge extraction, specialist knowledge extraction and guideline knowledge extraction.

3. The gout diagnosis and treatment system for combination of TCM and WM to claim 1, wherein the predictive reasoning module is configured to perform offline learning training, similar case presentation, staged diagnostic output and TCM prescription recommendation.

4. The gout diagnosis and treatment system for combination of TCM and WM according to claim 1, wherein the evaluation feedback module is configured to perform specialist follow-up diagnostics feedback and treatment effect evaluation feedback.

5. The gout diagnosis and treatment system for combination of TCM and WM according to claim 1, wherein the data storage module is configured to perform knowledge graph storage, training model storage and original data storage.

6. The gout diagnosis and treatment system for combination of TCM and WM according to claim 1, wherein the knowledge extraction module comprises:
a model layer, configured to define an ontology type by TCM specialists; wherein the ontology type comprises named entity classification and entity relationship classification;
a data layer, configured to perform manual annotation on extracted electronic medical record data to obtain annotated samples, and perform automatic annotation on electronic medical records by using the annotated samples and a sequence annotation algorithm to identify entities and entity relationships in medical materials, and store the entities and the entity relationships into the database.

7. The gout diagnosis and treatment system for combination of TCM and WM according to claim 1, wherein the predictive model of the predictive reasoning module is configured to divide a gout course to obtain gout course stages.

8. The gout diagnosis and treatment system for combination of TCM and WM according to claim 1, wherein in the offline training stage of the predictive reasoning module, the predictive model is obtained by training a data set containing input symptoms, corresponding syndrome elements of disease nature, and corresponding syndrome elements of disease locations;
in the online use stage, the predictive reasoning module is configured to receive patient data, and extract information for determining the gout course stage comprising a serum urate concentration, locations and numbers of joint swelling and pain and extract information for the symptoms of the patient by using keyword, synonym matching and semantic understanding technology, evaluate the gout course stage based on the extracted information, and match a basic diagnosis and treatment plan for the gout course stage; the predictive reasoning module is configured to use the predictive model to predict syndrome elements of disease nature and disease locations according to the symptoms of the patient, search from the knowledge graph according to the predicted syndrome elements of the disease nature and the disease locations to obtain TCM drugs; and form the recommendatory treatment plan by combining the basic treatment plan and the TCM drugs.
